(19) United States
(12) Patent Application Publication
(10) Pub. No.: US 2020/0353250 A1
(43) Pub. Date: Nov. 12, 2020

(54) ADAPTIVE TREATMENT MANAGEMENT SYSTEM

(71) Applicant: Medtronic, Inc., Minneapolis, MN (US)

(72) Inventors: Tarek Haddad, Minneapolis, MN (US); Vinod Sharma, Maple Grove, MN (US)

(21) Appl. No.: 16/863,340

(22) Filed: Apr. 30, 2020

Related U.S. Application Data
(60) Provisional application No. 62/844,697, filed on May 7, 2019.

(51) Int. Cl. A61N 1/362; A61N 1/37; A61N 1/365; A61N 1/372; G16H 10/60; G16H 20/17; G16H 40/67; G16H 50/30

(52) U.S. Cl. CPC A61N 1/3629; A61N 1/3706; A61N 1/36585; G16H 50/30; G16H 10/60; G16H 20/17; G16H 40/67; A61N 1/37282

(57) ABSTRACT

An adaptive treatment management system includes a sensor system, a treatment optimization system, and a treatment delivery system. The treatment optimization system may update a treatment regimen multiple times between physician visits to facilitate optimization of treatment. The treatment optimization system may determine a treatment regimen based on at least one of patient data, treatment compliance data, and related treatment data. Patient data may be provided by the sensor system. Treatment compliance data may be provided by the treatment delivery system. Related treatment data may be provided by a remote system.

This application claims the benefit of U.S. Patent Application No. 62/844,697, filed May 7, 2019, which is incorporated herein by reference in its entirety.

The present technology is generally related to medical treatment. In particular, the present technology is related to managing treatment for heart failure or other chronic condition.

As a specific example of chronic condition, congestive heart failure (CHF) is a serious condition that occurs when a heart is unable to consistently pump blood at an adequate rate. To improve the ability of the heart to efficiently pump blood, CHF patients may require an implantable medical device (IMD). IMDs such as implantable cardioverter defibrillators (ICDs) or pacemakers are capable of delivering cardiac resynchronization therapy for improving a CHF patient's heart function. Despite using IMDs to improve heart function, CHF patients may progressively deteriorate, as evidenced by weight gain, dyspnea, orthopnea, change in blood pressure, malaise, fatigue, peripheral edema (swelling in legs and feet), fainting, and/or palpitations.

Patient data may be obtained in a variety of ways. Typically, a patient directly conveys health data to medical personnel during an office visit. Some data may be automatically generated and sent over the internet to computer systems or health care systems. For example, electronic weight scales are configured to weigh a patient and then automatically transmit that data to the health care system.

In response to the collected data, healthcare systems can respond in a variety of ways. Some healthcare systems can generate health alerts based upon data detected by an IMD. One exemplary healthcare system described in U.S. Patent Publication No. 2010/0030293 to Sarkar et al. generates alerts for a patient to seek medical treatment in response to detected information. A medical device may detect worsening heart failure in the patient based on a diagnostic parameter. Upon detecting worsening heart failure, the medical device may, for example, provide an alert that enables the patient to seek medical attention before experiencing a heart failure event. While numerous healthcare systems can automatically notify health care workers of potential health issues, a healthcare system typically requires a physician's input to adjust therapy (e.g., medication) delivered to a patient.

### SUMMARY

The techniques of this disclosure generally relate to an adaptive treatment management system that can administer different treatment regimens to maintain a patient in a steady and stable condition between physician visits, even when the patient is not at a high risk of hospitalization, for chronic conditions, such as heart failure, chronic obstructive pulmonary disease (COPD), or renal dysfunction. The system may update a treatment regimen multiple times between physician visits to facilitate optimization of treatment, which may attempt to minimize patient risk scores, to minimize deviance from physician-prescribed treatment regimens, and to minimize patient symptoms.

In one aspect, the present disclosure provides a treatment management system comprising a sensor system to provide patient data about a patient or an environment related to the patient. The treatment management system also comprises a treatment delivery system to administer treatment based on a treatment regimen and to provide treatment compliance data. The treatment management system further comprises a treatment optimization system operably coupled to the sensor system and the treatment delivery system to update a treatment regimen based on the patient data from the sensor system and the treatment compliance data from the treatment delivery system and to provide the updated treatment regimen to the treatment delivery system.

In another aspect, the present disclosure provides a treatment optimization system comprising a data communication interface operably couplable to a sensor system configured to provide patient data and a treatment delivery system configured to administer treatment based on a treatment regimen. The treatment optimization system also comprises a memory configured to store data representing a risk score generator and a treatment regimen generator and a processor operably coupled to the data communication interface and the memory. The processor is configured to: update a patient score in response to administering a previous treatment based on a previous treatment regimen, wherein the patient score is based on at least one score selected from the one or more risk scores, one or more treatment scores, and one or more symptom scores; determine a treatment regimen in response to the updated patient score and the previous treatment regimen; and provide the treatment regimen to the treatment delivery system.

In another aspect, the present disclosure provides a treatment optimization system comprising a data communication interface operably couplable to a sensor system configured to provide patient data and a treatment delivery system configured to administer treatment based on a treatment regimen. The communication interface is configured to receive data representing a predetermined physician-limited parameter region. The treatment optimization system also comprises a memory configured to store the data representing the predetermined physician-limited parameter region and a processor operably coupled to the data communication interface and the memory. The processor is configured to: determine a patient score based on at least one of a risk score, a treatment score, or a symptom score using the patient data; determine a treatment regimen based on the patient score; determine whether the treatment regimen is within the predetermined physician-limited parameter region; and provide the treatment regimen to the treatment delivery system in response to determining that the treatment regimen is within the predetermined physician-limited parameter region to administer treatment based on the treatment regimen.

In yet another aspect, the present disclosure provides a treatment optimization system comprising a data communication interface operably couplable to a sensor system configured to provide patient data and a treatment delivery system configured to administer treatment based on a treatment regimen. The communication interface is configured to receive a physician-based treatment regimen. The treatment optimization system also comprises a memory configured to store data representing the physician-based treatment regimen and a processor operably coupled to the data communication interface and the memory. The processor is configured to: determine whether one or more risk scores are in a stable region representing a stable patient after administering treatment according to a current treatment regimen; determine a treatment score based on a difference between the current treatment regimen and the physician-based treatment regimen in response to the one or more risk scores being in the stable region; determine an updated treatment regimen based on the treatment score; and provide the updated treatment regimen to the treatment delivery system.

In still another aspect, the present disclosure provides a treatment delivery system comprising a plurality of receptacles. Each receptacle is to retain a different drug or a different dose. The treatment delivery system also comprises one or more cartridges. Each cartridge is to contain a different drug. Each cartridge comprises a different drug identifier associated with a different drug or different dose to load into a respective receptacle. The treatment delivery system further comprises a controller configured to detect each drug identifier of the one or more cartridges and to transmit a request over a data communication interface to deliver a new cartridge associated with a specific drug identifier in response to an updated treatment regimen.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a block diagram that illustrates one example of a treatment management system according to the present disclosure.

FIG. 2 is a conceptual diagram that illustrates one example of an environment usable with the treatment management system of FIG. 1.

FIG. 3 is a flow diagram that illustrates one example of a method of using the treatment management system of FIG. 1.

FIG. 4 is a state diagram that illustrates one example of a method of implementing the optimizing treatment method of FIG. 3 using multiple generators.

FIG. 5 is a flow diagram that illustrates one example of a method for determining the patient score usable in the method of FIG. 3.

FIG. 6 is a flow diagram that illustrates one example of implementing the optimizing treatment method of FIG. 3 using corroboration of risk scores.

FIG. 7 is a flow diagram that illustrates one example of implementing the optimizing treatment method of FIG. 3 integrating physician input.

FIG. 8 is a conceptual diagram that illustrates one example of a physician-limited parameter region usable in the method of FIG. 7.

FIG. 9 is a conceptual diagram that illustrates one example of a treatment delivery system usable with the treatment management system of FIG. 1.

FIG. 10 is a conceptual diagram that illustrates one example of managing treatment of a patient using the method of FIG. 3.

FIG. 11 is a conceptual diagram that illustrates another example of managing treatment of a patient using the method of FIG. 3.

### DETAILED DESCRIPTION

The techniques of this disclosure generally relate to an adaptive treatment management system that can administer different treatment regimens to maintain a patient in a steady and stable condition between physician visits, even when the patient is not at a high risk of hospitalization, for chronic conditions, such as heart failure, chronic obstructive pulmonary disease (COPD), or renal dysfunction. The system may update a treatment regimen multiple times between physician visits to facilitate optimization of treatment, which may attempt to minimize patient risk scores, to minimize deviance from physician-prescribed treatment regimens, and to minimize patient symptoms.

The present techniques may provide a dynamic treatment that manages physician-guided titration of medication based on a patient risk status, treatment adjustments for daily indiscretions, provides a reduction in the total amount of diuretic a patient takes, efficiently obtains data from non-implantable or non-wearable sources, facilitates patient compliance with a treatment regimen, reduces complexity of a treatment regimen, detects non-compliance, allows for efficient triaging, collects data about the outcome of treatment, and automatically schedules nurse and doctor appointments.

As used herein, the term “or” is generally employed in its inclusive sense, for example, to mean “and/or” unless the context clearly dictates otherwise. The term “and/or” means one or all the listed elements or a combination of at least two of the listed elements.

The phrases “at least one of” and “one or more of” followed by a list conjoined by “and” or “or” generally refers to any one of the items in the list and any combination of two or more items in the list unless the context clearly dictates otherwise.

Reference will now be made to the drawings, which depict one or more aspects described in this disclosure. However, it will be understood that other aspects not depicted in the drawings fall within the scope of this disclosure. Like numbers used in the figures refer to like components, steps, and the like. However, it will be understood that the use of a reference character to refer to an element in a given figure is not intended to limit the element in another figure labeled with the same reference character. In addition, the use of different reference characters to refer to elements in different figures is not intended to indicate that the differently referenced elements cannot be the same or similar.

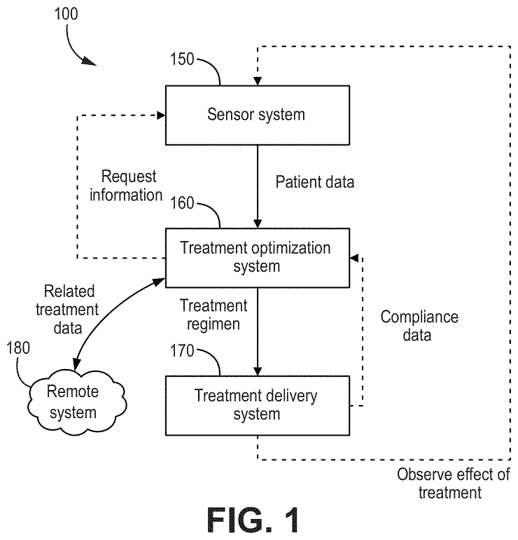

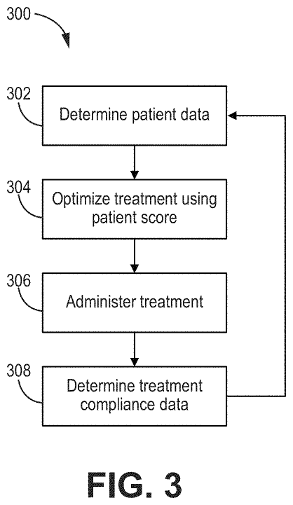

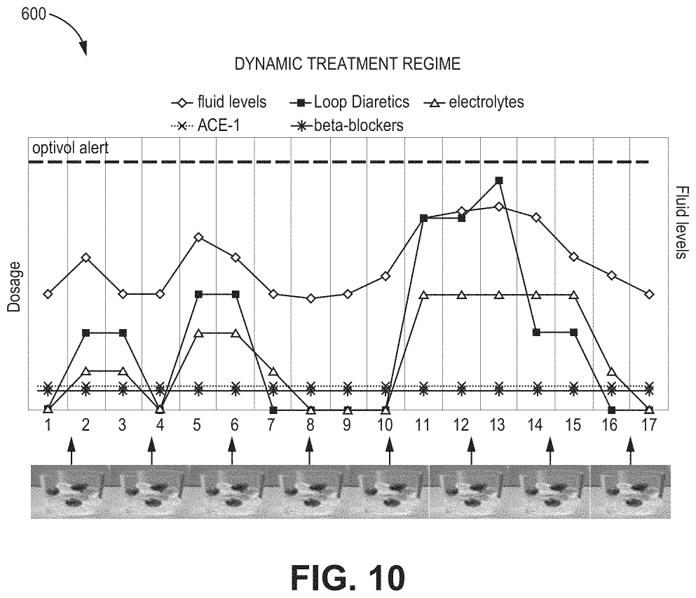

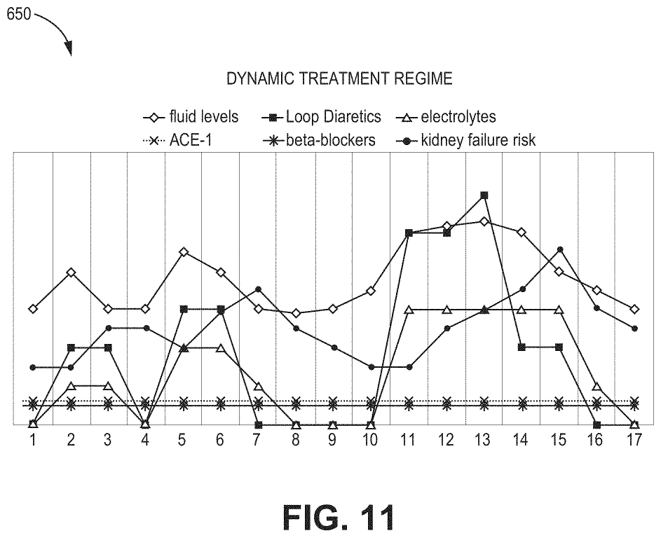

FIGS. 1-2 and 9 show a treatment management system and various components for managing treatment of a patient. FIGS. 3-8 show various diagrams of techniques that may be used by, or with, the treatment management system. FIGS. 10-11 show various examples of managing treatment using the treatment management system of FIGS. 1-2 and 9 with one or more of the techniques shown in FIGS. 3-8.

FIG. 1 is a block diagram that illustrates one example of a treatment management system according to the present disclosure. As illustrated, a treatment management system 100 may include a sensor system 150, a treatment optimization system 160, a treatment delivery system 170, and a remote system 180. The treatment management system 100 may be used to administer treatment to a patient based on a treatment regimen for the patient and update the treatment regimen on a regular basis, or as needed, between physician visits.

The treatment management system 100 may update the treatment regimen based on the frequency of administering treatment. For example, the treatment regimen may prescribe administration of certain treatments one or multiple times daily, such as three times daily, and the treatment regimen may be updated at least daily or at the highest frequency of administration, such as three times daily, or at any suitable frequency sufficient to keep the patient in a steady, stable, or steady and stable condition. In some embodiments, treatment may be adjusted, or titrated, upon updating of the treatment regimen by the treatment management system 100. For example, a physician may provide a prescription that may map doses and types of medication to a risk score or other patient status, and the treatment management system 100 may determine and provide a treatment regimen based on the risk score, or changes in risk score, on a daily, 2-day, 3-day, weekly, or any suitable time basis.

The sensor system 150 may include one or more sensors to detect various parameters related to a patient and an environment related to the patient. The detected parameters may be used to provide patient data or other data. Various examples of components of the sensor system 150 include a patient implantable sensor, a patient wearable sensor, an external sensor not worn by the patient, a graphical or audible user interface to accept user input, or a memory to store historical, or past, patient data.

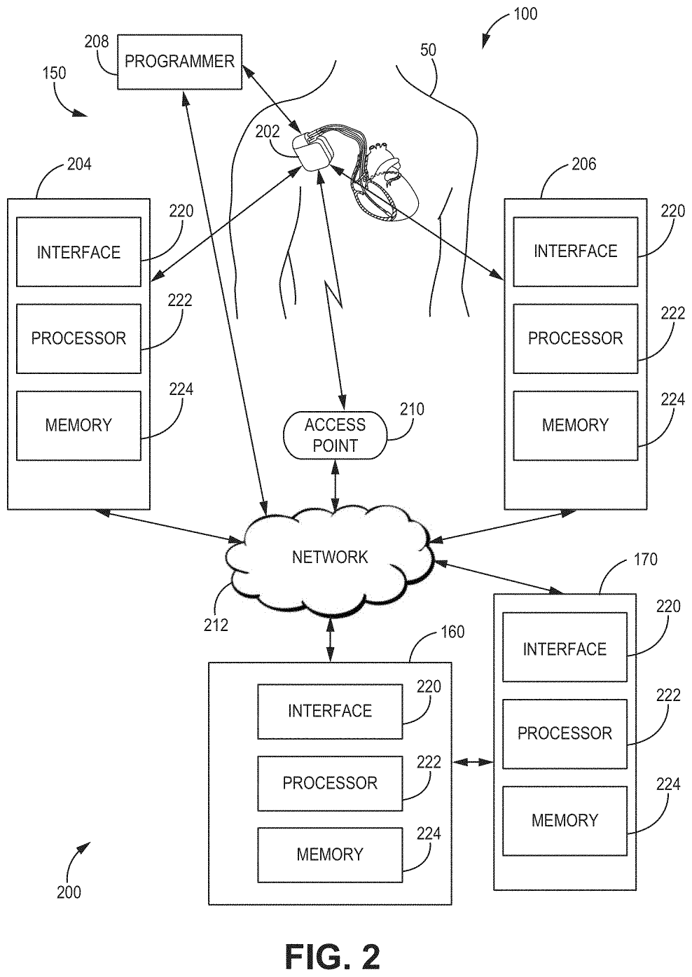

As used herein, the term “patient data” generally refers to data about a patient or an environment related to the patient. Various examples of sensors and devices usable to detect, collect, or determine, patient data are described herein, for example, with respect to FIG. 2. Non-limiting examples of patient data include, for example, fluid level, blood pressure, heartrate, heartrate variability, pulse transit time, QT interval, weight level, respiratory rate, respiratory effort, heart sounds, ejection fraction, potassium level, sodium level, creatinine level (or another renal biomarker such as blood urea nitrogen (BUN)), brain natriuretic peptide (BNP) or its precursor form NT-proBNP, pulmonary atrial pressure, jugular vein size, posture, patient activity, tissue perfusion level, oxygen saturation level, blood glucose level, arrhythmia (atrial or ventricular) incidence and burden (e.g., premature atrial or ventricular beats per day), COPD burden, and imaging of the patient's heart. Various specific sensors may be used to collect each type of patient data, such as a fluid level sensor, blood pressure sensor, etc. Patient data may include patient outcome data, such as whether a patient is hospitalized or other healthcare utilization information. Patient data may also include related information about the patient, such as other disease conditions or current treatments (e.g., diabetes, kidney failure, obesity, sleep apnea, dementia, Parkinson's, etc.).

The sensor system 150 may be operably coupled to the treatment optimization system 160 to provide patient data to the system 160. The treatment optimization system 160 may use the patient data to provide, or determine, a treatment regimen that is optimal for the patient. The optimized treatment may be described as treatment that is specific to the patient. In particular, the historical patient data may be used to determine a covariance with current patient data or to determine various risk factors based on patient background or history, for example, using artificial intelligence (AI) or other adaptive feedback methods. The treatment regimen may be determined based on at least one of patient data, compliance data, and related treatment data.

Some patient data may be automatically provided to the treatment optimization system 160, and other patient data may be provided to the treatment optimization system in response to a triggering event. In some embodiments, the treatment optimization system 160 may request information from the sensor system 150, which may be described as additional patient data used to supplement the previously received, or initial, patient data. In particular, the treatment optimization system 160 may request information to confirm, or corroborate, information in the initial patient data (e.g., “Are you breathless?” or “Did you go to the hospital?”). For example, patient data indicating high blood pressure, or hypertension, may be corroborated, or confirmed, using an eye scan from an imaging sensor. The additional patient data may be described as corroborating, confirming, or adjacent patient data. The additional patient data may be collected by the sensor system 150 or the treatment optimization system 160. Various examples of additional patient data requested by the treatment optimization system 160 are described herein with respect to FIG. 2.

As used herein, “compliance data” or “treatment compliance data” generally refers to data associated with the patient receiving the administered treatment. The compliance data may be sufficient to determine whether treatment was dispensed to the patient or whether the patient actually received treatment. Various sensors and devices usable to detect, collect, or determine, compliance data are described herein with respect to FIG. 2. Compliance data may be used by the treatment optimization system 160 for training one or more algorithms, or generators, to produce subsequent, or future, treatment regimens.

Non-limiting examples of compliance data include, for example, medication compliance (e.g., did patient take correct medication in the rise dose in a timely manner), nutrition compliance (e.g., restricting salt and water intake), physical activity compliance (e.g., exercise), and refraining from substance usage (e.g., smoking). Compliance data may also include data to confirm the identity of a user (e.g., to facilitate identification of medicine user) or that the user has actually complied with the treatment regimen, such as facial recognition data, voice recognition data, fingerprint data, verbal confirmation data, internal medication sensor data, dispenser confirmation data (e.g., to confirm what was dispensed for medicine or nutrition), and motion sensor data (e.g., to confirm exercise).

In the illustrated embodiment, the treatment optimization system 160 may be operably coupled to the treatment delivery system 170 to receive compliance data. The compliance data may be received, for example, at the same or similar frequency used to update the treatment regimen or to administer treatment or at any other suitable rate to manage treatment in a steady or stable region.

As used herein, the term “related treatment data” generally refers to data related to one or more disease conditions and treatments in the treatment regimen. Related treatment data may include information about a particular treatment that has been used for other patients who share the same or similar characteristics as the current patient. For example, the patient may be undergoing treatment for both heart failure and chronic obstructive pulmonary disease (COPD), and the related treatment data may contain, or be based on, information about treatments used for other patients undergoing the same treatments. Related treatment data may be used by the treatment optimization system 160 for training one or more algorithms, or generators, to produce subsequent, or future, treatment regimens. For example, an initial treatment regimen may be determined by the treatment optimization system 160 based on related treatment data, which may include large population level data for the same or similar disease condition or treatment, with little or no patient data specific to the patient. Patient data may then be used to further update the treatment regimen.

In the illustrated embodiment, the treatment optimization system 160 may be operably coupled to the remote system 180 to receive related treatment data. In particular, the treatment optimization system 160 may be operably coupled to the remote system 180 over the internet. In some embodiments, the treatment optimization system 160 may be operably coupled to the sensor system 150 or the treatment delivery system 170 over the internet, and the functionality of the remote system 180 may be integrated into the treatment optimization system, for example, as a single unit. A treatment optimization system 160 deployed on the remote system 180 may be able to live as a dynamic database that is constantly growing as the number of patients utilizing it, and the duration over which the patients use the system, increases.

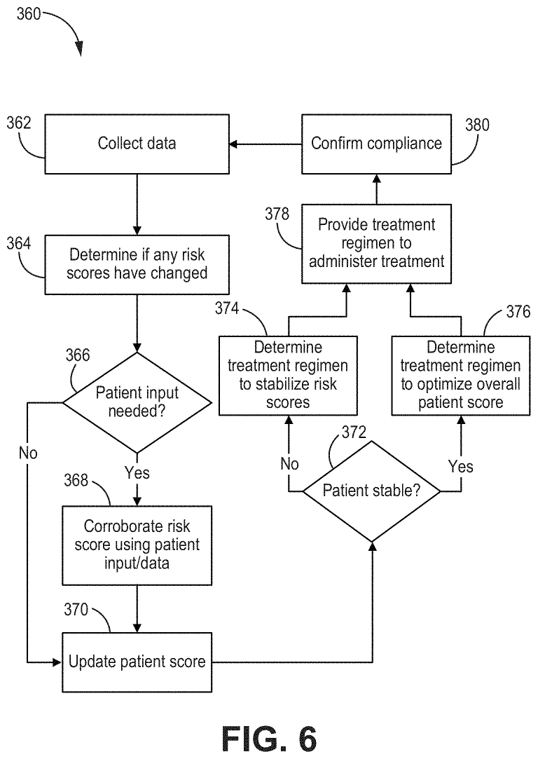

The related treatment data may be received at any suitable rate to manage treatment in a steady or stable region as discussed herein with respect to FIG. 6. In some embodiments, the related treatment data may be received during generation of a first, or initial, treatment regimen and periodically at a slower frequency relative to updating the treatment regimen or administering treatment. In other embodiments, the related treatment data may be received and used to update the treatment regimen in real-time or at the same or similar frequency as updating the treatment regimen or administering treatment.

The treatment optimization system 160 may be operably coupled to the treatment delivery system 170 to provide the treatment regimen. The treatment delivery system 170 may use the treatment regimen to administer treatment to the patient.

As used herein, to “administer treatment” refers to providing information to the patient to take a treatment (such as a notification), to make treatment available to the patient (such as dispensing a drug), or to control a device or system to automatically provide treatment to the patient (such as a drug delivery pump or an oxygen machine to treat COPD). Examples of drug delivery pumps that may be used to automatically provide treatment to the patient include a diuretic pump or an insulin pump depending on the disease condition.

The treatment management system 100 may be described as a “closed-loop” treatment management system, for example, due to the feedback provided to and between various systems 150, 160, 170, 180 of the overall system 100. In one example, the compliance data from the treatment delivery system 170 may be provided to the treatment optimization system 160 to confirm, or corroborate, the administration of treatment to the patient. In another example, the sensor system 150 may be used to observe the effect of the administered treatment, for example, after the treatment delivery system 170 is used to administer treatment. Other forms of feedback are also contemplated in the use of the treatment management system 100.

FIG. 2 is a conceptual diagram that illustrates one example of an environment usable with the treatment management system of FIG. 1. As shown, an environment 200 for use with the treatment management system 100 may include the body of a patient 50.

Any suitable components, or device, may be used to implement the system of treatment management system 100. Non-limiting examples of systems usable in the treatment management system 100 are described in U.S. patent application Ser. No. 16/394,942, filed Apr. 25, 2019, published as U.S. Patent Application No. 2019/0329043, and U.S. patent application Ser. No. 15/402,839, filed on Jan. 10, 2017, published as U.S. Patent Publication No. 2017/0245794, which are incorporated by reference into this disclosure. In some embodiments, the treatment management system 100 is configured to seamlessly trigger the adjustment of a patient's treatment regimen, or treatment plan, without directly communicating with the patient's physician any time after the prescribed treatment regimen has been sent to a centralized communication center for storage or stored into a memory of a computing device.

The treatment regimen may comprise one or more rounds of treatment (e.g., a first round of medication, a second round of medication, etc.). In general, adjusting the treatment regimen of the patient may depend on various factors, such as one or more risk scores, which may be determined based on patient data from one or more devices of the sensor system 150.

The sensor system 150 may include any suitable device for acquiring patient data. In some embodiments, the sensor system 150 may include a patient implantable device 202, such as an implantable medical device (IMD), having a patient implantable sensor, a patient wearable device 204 having a patient wearable sensor, and an external device 206 having an external sensor.

Various types of patient implantable sensors may be used to operably couple to circuitry of the patient implantable device 202 for use in providing patient data. Non-limiting examples of patient implantable sensors include an implantable electrical sensor with one or more electrical contacts (e.g., electrodes), a biochemical sensor, a motion sensor (e.g., 3-axes accelerometer), a piezoelectric sensor, an optical sensor, a temperature sensor, a geo-positioning sensor (e.g., GPS sensor), or a microphone. The electrical contacts may be used to provide electrical stimulation to tissue of the patient, such as cardiac tissue or kidney tissue.

One or more patient implantable sensors may be incorporated into, or operably coupled to, various implantable medical devices 202. Non-limiting examples of implantable medical devices 202 include a leaded or leadless pacemaker, an implantable cardioverter defibrillator (ICD), a cardiac resynchronization device with or without defibrillation capability (CRT or CRT-D), a leaded or leaded monitoring device, or an extravascular implantable cardioverter defibrillator (EVICD).

Various types of patient wearable sensors may be used to operably couple to circuitry of the patient wearable device 204 for use in providing patient data. Non-limiting examples of patient wearable sensors include an external electrical sensor with one or more electrical contacts (e.g., electrodes), a biochemical sensor, a motion sensor (e.g., 3-axes accelerometer), a piezoelectric sensor, an optical sensor, a temperature sensor, a geo-positioning sensor (e.g., GPS sensor), or a microphone.

One or more patient wearable sensors may be incorporated into, or operably coupled to, various patient wearable device 204. Non-limiting examples of patient wearable devices 204 include a heartrate monitor, a smartwatch, a perfusion sensor (e.g., a pulse oximeter), a patch to monitor vitals and monitor activity, a hearing aid with capability to detect activity and vitals (e.g. core temperature), or pendant-like devices to measure activity.

Various types of external sensors may be used to operably couple to circuitry of the external device 206 for use in providing patient data. Non-limiting examples of external sensors include an imaging sensor, a weight scale, a pressure sensor, or microphone.

One or more external sensors may be incorporated into, or operably coupled to, various external devices 206. Non-limiting examples of external devices 206 include a smartphone, a tablet, a personal computer, a home security camera, a treatment dispenser, a weight scale, a blood pressure cuff, a bed sensor to measure sleep metrics such as number of times getting out of bed and restlessness, or furniture.

The various sensors of the sensor system 150 may be used in various ways to provide patient data. In one example, a pressure sensor may be embedded into furniture, such as a couch or bed, and used to provide respiratory data and heart rate.

In another example, an electrical sensor on the patient implantable device 202 or a patient wearable device 204 may include an electrical sensor used to monitor electrical activity to provide, for example, an electrocardiogram (ECG), an impedance value, respiration data, or a fluid level.

In another example, a 3-axis accelerometer can be used to measure patient's activity, changes in gait pattern, and frailty metrics (e.g., time required to get up from a couch and walk certain distance). An accelerometer and/or piezo electric sensor can be used to measure S3 and S4 heart sounds (these sounds may emerge as a patient gains fluid and their heart failure begins to get worse). A temperature sensor can be used to measure diurnal variation in temperature and assess infection risk.

One or more devices of the sensor system 150 may be operably coupled to a programmer 208 or an access point 210. The programmer 208 or access point 210 may be wirelessly connected or connected by wire to one or more devices. For example, the programmer 208 and the access point 210 may be wirelessly connected to the patient implantable device 202 to transmit and receive data. In turn, the programmer 208 and the access point 210 may be operably coupled to a network 212, which may include a local network, wide area network, or the internet, to further transmit and receive data.

The treatment delivery system 170 may include any suitable device for administering treatment for the patient. In some embodiments, the treatment delivery system 170 may include a drug dispenser to contain one or more drugs, an automated subcutaneous (SubQ) or fully implanted treatment pump, an intravenous or intraperitoneal line, or a graphical or audible user interface to provide treatment information to the patient. The treatment delivery system 170 may be operably coupled to one or both of the network 212 and the treatment optimization system 160.

Drug dispensers may be used in some treatment delivery systems 170. Some drug dispensers may be configured to include two or more different receptacles, or compartments, for holding two or more different medications or different dosages of the same medication (e.g., diuretics, hypertensive medicine, etc.). Drug dispensers may be external, wearable, or implantable to administer the proper dosage of medication according to the treatment regimen.

Some drug dispensers include two or more compartments, such as a PILLO™ automated pill dispenser from Pillo, Inc. of Boston, Mass., to release medication into a single container for patient use, and some drug the dispensers release medication directly into the body, for example, as described in U.S. Pat. Nos. 7,001,359, 7,054,782, 7,008,413, 7,264,611, and 7,160,284, which are incorporated by reference into this disclosure. One example of an implantable drug dispenser is a subcutaneous drug dispenser (e.g., subcutaneous implantable devices such as a drug delivery, such as the MiniMed PARADIGM® REVEL′ insulin pump from Medtronic plc of Dublin, Ireland, or an SC2 infuser from SC Pharmaceuticals of Burlington, Mass., or subcutaneous device such as OmniPod® insulin management system from Insulet Corporation of Acton, Mass.).

Drug dispensers may be configured to receive a treatment regimen via communication signals through a data communication interface, which may include a transceiver or transmitter. In some embodiments, the data communication interface may utilize a wireless communication protocol, such as BLUETOOTH™ technology.

In some embodiments, the drug dispenser may be configured to receive commands to adjust a dosage or type of drug to be administered. In some embodiments, the drug dispenser may be configured to signal another implantable medical device (LINQ™, pacemaker, etc.) or an external device (e.g., a smartphone) to provide information (e.g., drug level is depleting or running very low).

The drug dispenser may be configured to receive instructions to dispense, or otherwise administer, to facilitate ensuring that the patient has access to the correct medication and/or dosage of medication. Once the treatment optimization system 160 determines one or more medications for a treatment regimen, the treatment optimization system 160 may automatically signal the treatment delivery system 170 to administer medication according to the updated treatment regimen. The treatment optimization system 160 may signal the treatment delivery system 170 to adjust the dosage delivered to a patient, for example, to increase or reduce the dosage based on the updated treatment regimen.

The treatment delivery system 170 may automatically switch from a first to a second dosage compartment for drug delivery. The drug dispenser, or treatment delivery device, may rotate from the first dosage compartment that stores a first dosage to the second dosage compartment that stores a second dosage for delivery to the patient. The treatment delivery device may or may not automatically notify the patient that the treatment regimen has been modified. The treatment delivery device may automatically notify the patient to take the medication during the day. The treatment, or drug, may be automatically dispensed to the patient at the proper dosage. The treatment delivery device may be set to automatically lock the mechanism for drug delivery once the proper dosage has been delivered.

The treatment optimization system 160 may include any suitable device for optimizing treatment and determining a treatment regimen for the patient. In some embodiments, the treatment optimization system 160 may be integrated with the treatment delivery system 170 or located on a server connected through the network 212 with other parts of the treatment management system 100.

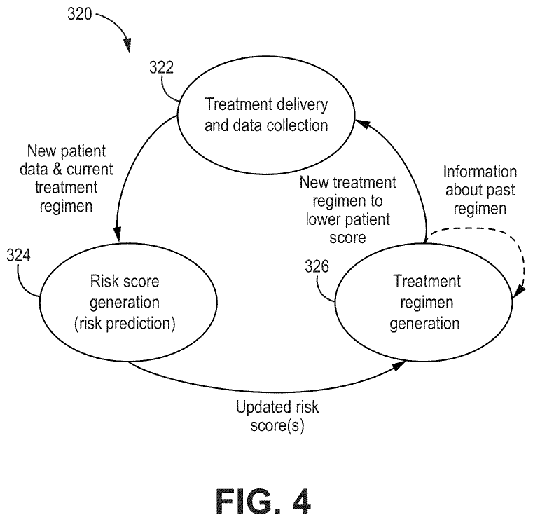

A treatment delivery system 170 that can control the amount of treatment administered to the patient may provide robust feedback in the form of treatment compliance data. In some embodiments, the treatment delivery system 170 may not be able to control the amount of treatment administered but may be able to provide treatment compliance data indicating whether the patient has at least accessed and, in some cases received, the treatment. For example, the treatment delivery system 170 may simply include a medication container that can indicate whether the container has been opened. For example, a container may include a wireless tag that can provide a signal to a smartphone or other device, such as smart speaker, when the container has been opened or each time the container is opened. Such a treatment delivery system 170 may be used, for example, when the patient is on vacation and an override mode is active, which is described herein with respect to FIG. 4.

Some treatment delivery systems 170 may include a graphical or audible user interface to provide treatment information to the patient, such as dosage or other treatment information. In one example, the graphical or audible user interface may suggest to the patient to avoid salts for the day due to an elevated risk score.

One or more systems of the treatment management system 100 may include one or more computing devices having a data communication interface 220, a processor 222, and a memory 224. In particular, one or more of the systems may include one or more controllers, sensors, or interfaces, each of which may include the processor 222, such as a central processing unit (CPU), computer, logic array, processing circuitry, or other device capable of directing data coming into or out of the system. Each system may include circuitry used to couple various components of the controller together or with other components operably coupled to the processor 222. The functions of the processor 222 may be performed by hardware and/or as computer instructions on a non-transient computer readable storage medium.

The processor 222 may include any one or more of a microprocessor, a microcontroller, a digital signal processor (DSP), an application specific integrated circuit (ASIC), a field-programmable gate array (FPGA), and/or equivalent discrete or integrated logic circuitry. In some examples, the processor 222 may include multiple components, such as any combination of one or more microprocessors, one or more controllers, one or more DSPs, one or more ASICs, and/or one or more FPGAs, as well as other discrete or integrated logic circuitry. The functions attributed to the controller or processor 222 herein may be embodied as software, firmware, hardware, or any combination thereof. While described herein as a processor-based system, an alternative controller could utilize other components such as relays and timers to achieve the desired results, either alone or in combination with a microprocessor-based system.

In one or more embodiments, the exemplary systems, methods, techniques, and other related functionality may be implemented using one or more computer programs using a computing apparatus, which may include one or more processors 222 and/or memory 224. Program code and/or logic described herein may be applied to input data/information using the data communication interface 220 to perform functionality described herein and generate desired output data/information. The output data/information may be applied as an input to one or more other devices and/or methods as described herein or as would be applied in a known fashion using the data communication interface 220. In view of the above, it will be readily apparent that the controller or processor functionality as described herein may be implemented in any manner known to one skilled in the art.

The network 212 may generally be used to transmit information or data (e.g., patient data such as physiological data, risk level data, recovery data) between some or call devices of the treatment management system 100. However, network 212 may also be used to transmit information to other external computing devices (e.g., a CARELINK® system commercially available from Medtronic plc).

Typically, a wireless connection may be employed. In one example, the patent implantable device 202, the patient wearable device 204, the external device 206, the programmer 208, the access point 210, the treatment optimization system 160, and the treatment delivery system 170 may be interconnected, and able to communicate with each other, directly or through network 212.

The various systems of the treatment management system 100 may include one or more servers, computers, weight scales, portable blood pressure machines, biometric data collecting device, a computer, a symptom assessment system, a personal digital assistant (e.g., cell phone, tablet, or the like). In some examples, various devices of the treatment management system 100 may generate data to perform any of the various functions or operations described herein, e.g., generate a heart failure risk status based on the patient metric comparisons or create patient metrics from the raw metric data.

Heart failure (HF) risk status can be calculated in a number of ways known to a person of skilled art having the benefit of this disclosure. One example of calculating a risk score, or risk status, is described in U.S. Patent Publication No. 2019/0069851, filed Aug. 31, 2018, and U.S. Patent Publication No. 2019/0125273, filed Apr. 26, 2018, which are incorporated by reference in this disclosure.

Various data communication interfaces 220 may also include user interfaces. In some embodiments, one or more data communication interfaces 220 of the treatment management system 100 include input devices, such as a keyboard, a mouse, voice input, sensor for weight, etc., or output devices, such as a graphical or audible user interface (e.g., for touch screen inputs or voice commands), a printer, or other suitable means.

One or more processors 222 of the treatment management system 100 may be configured to perform some type of analog to digital conversion so that signal can be compared to some threshold. Each processor 222 may be configured to perform a variety of functions such as calculations, accessing data from memory performing comparisons, setting the start and end dates for each evaluation period, etc. The evaluation period serves as an evaluation window that encompasses data, acquired from each patient, that are within the boundaries (e.g., start and end times).

Various memory 224 of the treatment management system 100 may include volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital or analog media. In general, the memory 224 may store data, such as patient data, which may include heart failure patient data, heart failure prospective risk data, intracardiac or intravascular pressure, activity, posture, respiration, thoracic impedance, impedance trend, risk of hypervolemia or hypovolemia, etc. Evaluation period start and end times or dates may also be stored in the memory 224. In some cases, patient data may include data observations (e.g., data sensed from sensors that cross a threshold).

The programmer 208 may include any appropriate programming system, including one generally known to those skilled in the art, such as the Medtronic CARELINK™ programmer from Medtronic plc.

The treatment optimization system 160, for example, via the programmer 208, may send a request to a device of the sensor system 150, such as patient implantable device 202. The patient implantable device 202 may provide patient data (e.g., diagnostic information, real-time data related to absolute intrathoracic impedance that may be indicative of hypervolemia or hypovolemia, etc.) or diagnostic information selected by the request or based on already entered patient status to the programmer 208. The patient data may include patient metric values or other detailed information from the patient implantable device 202.

The patient data may be used to provide an alert or notification of the heart failure risk level by the treatment optimization system 160. The alert may be automatically transmitted, or pushed, when the heart failure risk level becomes critical. In addition, the alert may be a notification to a healthcare professional, e.g., a clinician or nurse, of the risk level and/or an instruction to patient 50 to seek medical treatment (e.g., testing to confirm worsening HF, etc.). In response to receiving the alert, the user interface may display the alert to the healthcare professional regarding the risk level or present an instruction to patient 50 to seek medical treatment.

Either in response to heart failure data, e.g., the risk level or patient metrics, or requested heart failure information, the user interface for a computing device or programmer 24 may present the patient metrics, the heart failure risk level, or recommended treatment (e.g., medication) to the user. In some examples, the user interface may also highlight the patient metrics that have exceeded respective metric-specific thresholds. In this manner, the user may quickly review those patient metrics that have contributed to the identified heart failure risk level.

Access point 210 may comprise a device that connects to network 112 via any of a variety of connections, such as telephone dial-up, digital subscriber line (DSL), or cable modem connections. In other examples, access point 210 may be coupled to network 112 through different forms of connections, including wired or wireless connections. In some examples, access point 210 may be co-located with patient 50 and may comprise one or more programming units and/or computing devices (e.g., one or more monitoring units) that may perform various functions and operations described herein.

In another example, access point 210 may be a LINQ™ device co-located within the patient and configured to sense, record and transmit data to network 112. Alternatively, a wearable patch such as a SEEQ™ device, configured for monitoring, may be attached to the skin of the patient. For example, the SEEQ™ device could be attached to the skin over the heart of the patient for cardiac monitoring. In another example, access point 210 may include a home-monitoring unit that is located within patient 50 and that may monitor the activity of IMD 16. LINQ™ and SEEQ™ devices commercially available from Medtronic plc may also be used as access point 210. Non-limiting examples of LINQ™ devices are described with respect to U.S. Publication No. 2016/0310031, filed Apr. 20, 2016, which is incorporated by reference in this disclosure.

The treatment optimization system 160 may include or be part of a centralized communication center staffed by one or more skilled nurses and an established workflow to manage/treat patients on a spectrum of episode severity (e.g., decompensated HF patient with hypotension versus normal blood pressure). The treatment optimization system 160 may be configured to perform complex computations for a large group of patients and may provide secure storage in memory for archival of information (e.g., patient metric data, heart failure risk levels, weight, blood pressure, etc.) that has been collected and generated from the sensor system 150 and the treatment delivery system 170.

FIG. 3 is a flow diagram that illustrates one example of a method of using the treatment management system of FIG. 1. As illustrated, a method 300 may include determining patient data 302. The patient data may be provided by a sensor system, which may include an implantable device, a wearable device, or an external device. Some patient data may be automatically provided, and other patient data may be provided to confirm a risk score.

A risk score may be used to determine a patient score. The method 300 may include optimizing treatment using the patient score 304. A treatment regimen may be generated, or determined, that reflects the optimized treatment. In general, the optimization of treatment will minimize the patient score, which typically corresponds to reduction of risks, treatment differences, and symptoms.

The method 300 may include administering the treatment 306, for example, based on the generated optimal treatment regimen. Administration of treatment may include providing a predetermined dosage of treatment for the patient to voluntarily take or automatically delivering the treatment (e.g., automated treatment pump into the patient's body or oxygen machine setting).

In addition, the method 300 may include determining treatment compliance data 308, for example, in response to administering the treatment. Treatment compliance data may provide some indication of whether the patient has complied with the administered treatment. For example, a camera disposed on or near a drug dispenser may verify the identity of a patient taking the medication that is dispensed, which may provide at least some level of verification of compliance to the treatment regimen by the patient.

Further, method 300 may return to determining patient data 302, optimizing treatment using patient score 304, and administering treatment 306, in an iterative loop or closed loop. The treatment may be optimized, or the treatment regimen may be updated, using the treatment compliance data. For example, the treatment compliance data may indicate whether the patient actually took the previous treatment regimen that was administered, which may be used to determine the appropriate next treatment regimen to administer. In particular, the patient score may be updated based on the treatment compliance data. For example, a risk score may change based on treatment compliance data as described herein with respect to FIG. 4.

FIG. 4 is a state diagram that illustrates one example of a method of implementing the optimizing treatment method of FIG. 3 using multiple generators. In particular, FIG. 3 shows the use of two generators. As illustrated, the method 320 may include a state of delivering treatment and collecting data 322. The collected data may include, for example, patient data, treatment compliance data, and related treatment data.

The method 320 may also include a state of risk score generation 324, or risk prediction, by a risk score generator, which may follow the treatment delivery and data collection 322. The risk score generator may be used to generate one or more risk scores based on collected data, such as new or updated patient data or data about the current treatment regimen. The risk score generator may be stored on a memory of the treatment optimization system.

A “risk” score refers to a measure that may indicate a decline in patient health. Non-limiting examples of a decline in patient health include an increased risk of hospitalization, an increased risk of death, or other indication of general health decline.

In general, the patient data may be used to determine one or more risk scores. For example, if a patient's blood pressure has increased, a risk score associated with high blood pressure (and an overall risk score) may also increase. The current treatment regimen may be used to adjust certain risk scores. For example, if a patient has been treated for high blood pressure by administering the highest allowable level of ACE inhibitor, the risk score associated with high blood pressure may increase even further. In another example, even if the patient's blood pressure has not changed from a high level, even after administering the highest allowable level of ACE inhibitor, the risk score associated with the high blood pressure may still increase.

The method 320 may also include a state of treatment regimen generation 326 by a treatment regimen generator. The treatment regimen generator may be used to determine a treatment regimen based on one or more risk scores, for example, provided by the risk score generator. As described herein, the treatment regimen generated may be within the bounds of a prescribed treatment regimen by a physician. The treatment regimen generator may also determine the treatment regimen based on other data and scores, such as a treatment score or a symptom score. The treatment regimen generator may be stored on a memory of the treatment optimization system. In some embodiments, the treatment regimen generator does not use patient data as direct input but rather determine the updated treatment regimen based on a patient score and past treatment regimen data. In general, the risk score generator and the treatment regimen generator are separate and different in that each has different inputs and different outputs than the other.

In general, the treatment regimen generator may generate the treatment regimen based on an overall patient score, which may be a combination of one or more scores. The treatment regimen generator may provide a new treatment regimen to lower, or minimize, the overall patient score. In general, as used herein, a low overall patient score is defined as corresponding to a better patient condition than a higher overall patient score. However, in some embodiments, a high overall patient score may be defined as corresponding to a better patient condition than a lower overall patient score.

The treatment regimen may also be generated based on information about one or more past treatment regimens. The past treatment regimen data may be used to update the patient score. For example, if a past treatment regimen to reduce blood pressure did not reduce a patient's high blood pressure, the patient score may increase and, accordingly, the new treatment regimen may increase a dosage of an ACE inhibitor or introduce a different treatment for high blood pressure.

In general, the treatment regimen generator may update a patient score in response to administering a previous treatment based on a previous treatment regimen, determine a treatment regimen in response to the updated patient score and the previous treatment regimen, and provide the treatment regimen to a treatment delivery system.

The method 320 may return to treatment delivery and data collection 322 after treatment regimen generation 326 in an iterative or “closed loop” fashion to continue delivering treatment, collecting data, generating risk scores, and updating treatment regimens.

In some embodiments, the treatment regimen generation 326 or treatment delivery and data collection 322 of the method 320 may enter into an override mode. In the override mode, the treatment regimen or treatment administration may be suspended in some manner. For example, the override mode may indicate that the treatment regimen should not be updated for a period of time or that treatment should not be administered for a period of time.

Various triggering events may initiate the override mode, which may be detected by one or more sensors or devices of the treatment management system. In some embodiments, physician input may trigger entry into the override mode, for example, when the patient is in the clinic or hospital, and the physician is administering treatment. The administration of treatment and the generation of updated treatment regimens may be suspended in an override mode. When the patient is ready to leave, physician input may trigger exiting from the override mode (e.g., disabling, turning off, etc.).

In some embodiments, patient input may trigger, or initiate, entry into the override mode, for example, when the patient is planning to be away from a home-based treatment delivery system on vacation or other trip. The administration of treatment by the home-based treatment delivery system and the generation of updated treatment regimens may be suspended, for example, because the patient does not have readily-available access to changes in dosage or different treatments while away from the home-based treatment delivery system. When the patient returns to the home-based treatment delivery system, patient input may trigger exit from the override mode.

In some cases, the treatment delivery system may be portable. The portable treatment delivery system may provide some or all the same functionality as a home-based treatment delivery system. In other words, the portable treatment delivery system may be capable of administering different treatments to the patient based on updated treatment regimens to at least some degree. For example, the portable treatment delivery system may include a limited number of different dosages of the same medication or a limited number of different medications. A patient may be informed by a smartphone, which may be considered a part of the portable treatment delivery system, to take a certain amount of medication from the portable treatment delivery system. In general, a portable treatment delivery system may allow the treatment management system to partially, or wholly, avoid or exit from the override mode.

A proximity sensor or other location-based sensor of the treatment management system may also be used to generate environmental data to trigger entry into or exit from the override mode, for example, instead of requiring manual physician input or manual patient input. In addition, data from other sources, such as GPS location from the patient's phone, may also be used to trigger entry into or exit from the override mode.

Further, in some embodiments, noncompliance with a treatment regimen may trigger the override mode or may trigger an increase in the overall patient score or one or more risk scores.

In some embodiments, the risk score generator or the treatment regimen generator may be updated based on related treatment data. For example, other patients undergoing the same or similar treatment may generate data that can be used to update various best practices with respect to risks or treatment. The generators may be updated, or trained, on a regular basis, for example, using updates or may even be updated in real-time. For example, the risk score generator or the treatment regimen generator may be stored on the cloud and used to generate risk predictions and treatments for one or more patients.

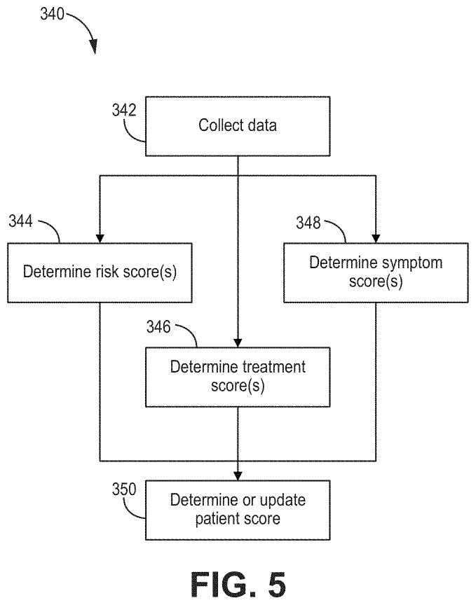

FIG. 5 is a flow diagram that illustrates one example of a method for determining the patient score usable in the method of FIG. 3. As illustrated, a method 340 may be used to determine or update a patient score 350 in response to collecting data 342. Although optimization of patient score and other scores are described generally in terms of minimization, the optimization of patient score using a maximum or target value is also contemplated in any suitable manner available to one skilled in the art having the benefit of this disclosure.

Based on the various types of data collected, one or more risk scores may be determined 344, each risk score representing a different parameter or metric of risk. One or more of the risk scores may be based on patient data, which may include corroborated patient data. In some embodiments, the risk scores may relate to potential heart failure risks.

Risk scores may represent input parameters or combinations of input parameters. A composite risk score may represent some or all input parameters. Non-limiting examples of risk scores, or input parameters to risk scores, include: an OPTIVOL™ score (e.g., impedance or impedance index representative of fluid level), a night heart rate (NHR) score (e.g., night ventricular rate score), a patient activity score, a heart-rate variability (HRV) score, an arrhythmia score (e.g., atrial tachycardia score or atrial fibrillation score), a ventricular rate score (e.g., mean rate during arrhythmia), a pacing score (e.g., ventricular pacing percentage for CRT), a shock score (e.g., number of shocks), respiration, tissue perfusion, temperature, a treated arrhythmia score, COPD risk, stroke risk, kidney failure risk, VT/VF risk, acute decompensation risk, high or low potassium risk, high or low glucose risk, or sleep apnea risk.

Various diagnostic metrics or metrics that may be used to determine a risk score can be extracted from patient data. Non-limiting examples of patient data include (1) an impedance trend index commercially available in IMDs from Medtronic plc, (2) intrathoracic impedance, (3) atrial tachycardia/atrial fibrillation (AT/AF) burden, (4) mean ventricular rate during AT/AF, (5) patient activity, (6) ventricular (V) rate, (7) day and night heart rate, (8) percent CRT pacing, or (9) number of shocks.

Some examples of medium- and high-risk calculations performed by the treatment optimization system are described in U.S. Patent Publication No. 2016/0361026, filed, Mar. 29, 2011, U.S. Patent Publication No. 2012/032243, filed Oct. 28, 2010, and U.S. Patent Publication No. 2019/0069851, filed Aug. 31, 2018, and U.S. Patent Publication No. 2019/0125273, filed Apr. 26, 2018, which are incorporated by reference in this disclosure.

Medium-risk status, for example, may involve one or more conditions such as AT/AF burden exceeding a threshold value (>6 hours/day), low % V pacing and high night heart rate (>85 bpm). High risk status, for example, may involve one or more conditions such as high OPTIVOL™/impedance index (>60 ohm-days), patient activity (<1 hour/day), high night heart rate (>85 bpm) and low HRV (<60 ms).

The impedance index may be an indicator of the amount of fluid congestion experienced by the patient. The impedance index is the difference between an impedance measured daily using, for example, the patient implantable device and a reference impedance, that can be continuously updated, established by the patient implantable device or during a visit to the physician. Non-limiting examples of determining and using impedance index are described in U.S. Pat. No. 7,986,994, granted Jul. 26, 2011, which is incorporated by reference in this disclosure.

Heart rate variability (HRV) may be a marker of autonomic tone and may provide prognostic information for mortality risk. A decrease in HRV is associated with increased sympathetic tone, or a higher risk score. Using HRV device diagnostic data, patients with low HRV (<100 ms) may be at a higher combined risk of death and hospitalization. Patients with HRV<50 ms may exhibit an even higher risk than those with HRV in the range of 50-100 ms.

Similar to HRV, elevated heart rate may be a marker of elevated sympathetic tone and has been shown to have prognostic value for worsening HF, or a higher risk score. Night Heart Rate (NHR), measured between midnight and 4 AM, can be a better metric than the day time heart rate. Day time heart rate can be affected by varying activity level (e.g., rest and exercise). Patients with high NHR (75±25 bpm) typically experience higher risk of being hospitalized or dying than those who had low NHR (73±11 bpm).

Additionally, declining patient activity is associated with worsening HF status and can potentially be of value for predicting HF hospitalization, which may correspond to a higher risk score. Declining patient activity can be determined by a variety of activity devices such as a FITBIT® device, cellphone or smartphone, etc.

Combination variables (e.g., combining pacing and arrhythmia related information) can also be used to evaluate worsening HF risk, or a higher risk score. For example, one of the components of combination variable is substantial decrease (>8%) in CRT pacing, which is associated with high HF events. A decline in CRT pacing can occur because of rapid conduction during AF. Thus, mean ventricular rate≥90 bpm and atrial fibrillation (AF) burden≥6 hours/day and shocks delivered to Ventricular Fibrillation/Ventricular Tachycardia (VT/VF) can also be components of the combination variable.

In general, the treatment management system may provide an updated treatment regimen to lower risk scores (or an overall risk score) and bring each of the variables within a steady and stable region, which may minimize the risk scores (or the overall risk score). In some embodiments, baseline medications may be maximized and diuretics may be lower to maintain physiological variables within a normal range.

One or more treatment scores may be determined 346 based on collected data each related to a different treatment. In particular, a treatment score may be determined based on a difference between a physician-based treatment regimen and the current treatment regimen being administered to the patient.

In some embodiments, treatment regimens may include a plurality of different treatments. Non-limiting examples of treatments that may be included in a treatment regimen include diuretics, heart failure medication (e.g. beta-blockers, ACE inhibitors, ARB, hydralazine, nitrates, ENTRESTO™ (sacubitril/valsartan) and mineralocorticoids), stroke medication, blood pressure medication (e.g., ACE inhibitors or beta blockers and calcium channel blockers), oxygen (e.g., for COPD), steroids (e.g., for COPD), physical activity (e.g., exercise), dietary recommendations, dietary supplements, sugar pills, oral insulin, Parkinson's/tremor medication, or anticoagulant medication.

A difference may be calculated for each of the treatments and incorporated into one or more treatment scores. The difference may also be described as a deviation from the physician-based treatment regimen. For example, the difference in treatment regimens may be driven by one or more of the other scores, such as a patient score or a symptom score, influencing the patient score and thus influencing the treatment regimen generated by the treatment optimization system. In some embodiments, when the risk scores and symptom scores are steady and stable, the current treatment regimen may be titrated toward the physician-based treatment regimen, which may minimize the treatment scores. The physician-based treatment regimen may be determined based on physician input and stored in a memory of the treatment management system.

One or more symptom scores may be determined 348 based on collected data representing one or more patient symptoms. In general, a symptom score may indicate other patient symptoms not reflected in the risk score. A symptom score may be determined based on how the patient feels, which may be independent of risk scores or treatment scores. For example, even though a patient may have low risk scores, the patient may have side effects from treatment that increases symptom scores. One example of a side effect is a patient may develop a cough from a beta blocker. The symptom scores may be based on patient data from sensor data or patient input. For example, a patient may input into a user interface of a smartphone a feeling of pain, which may be used to generate a symptom score. In response, the treatment management system may decrease, or otherwise change, one or more treatments to reduce patient symptoms, which may minimize the patient scores.

An overall patient score may be determined or updated 350 based on at least one of the score factors, which may include one or more risk scores, treatment scores, and symptom scores. The one or more score factors may be calculated and combined in any suitable manner to facilitate minimization of patient risks, treatment differences or deviations, and symptoms for the patient when generating treatment regimens.

One or more calculated risk scores may be weighted. A weighting function may be applied to one or more of the risk scores, which may be monotonic, non-linear, or both monotonic and non-linear. In one example, the weighting function may be an increasing monotonic, non-linear function.

The weighting function may be continuous and may include an inflection point that relates to a threshold or pseudo-threshold. For example, if a risk score increases past a certain threshold, the weighted risk score may dramatically increase, which may have the effect of prioritizing the risk score over other risk scores, treatment scores, or symptom scores that have not reached a similar threshold or pseudo-threshold. Each risk may have a different inflection point or pseudo-threshold, or a different risk profile altogether.

Various functions may also be applied to a treatment deviation to provide a treatment score or to symptom data to provide a symptom score. A weighting function may also be applied to one or more treatment scores or symptom scores.

Multiple scores may be combined using a summation. For example, multiple risk scores may be combined using a summation of multiple weighted risk scores.

The one or more risk scores, treatment scores, and symptom scores, whether weighted or not weighted, may be combined as inputs to a function that produces the overall patient score. The function may generally align the score factors such that minimization (or maximization) of the patient score generally balances the minimization of risk factors, treatment deviations, and patient symptoms. In one example, the scores may be summed.

FIG. 6 is a flow diagram that illustrates one example of implementing the optimizing treatment method of FIG. 3 using corroboration of risk scores. As illustrated, a method 360 may include collecting data 362, such as patient data, treatment compliance data, and related treatment data.

The method 360 may also include determining if any risk scores have changed 364, for example, based on the collected data. Risk scores may change based on the previous treatment provided or may stay the same.

Further, the method 360 may include determining whether patient input is needed 366. If patient input is needed, a request may be provided to the patient to provide more information (e.g., answering a question) or for action by the patient to collect additional data (e.g., step on a weight scale).

The method 360 may include corroborating a risk score using patient data 368, such as patient input, for example, in response to determining that patient input is needed 366. In one example, patient data may indicate a high risk-score related to blood pressure, and a patient may be asked to step in front of a camera to record an image of the patient's eye. The image of the patient's eye may be used to confirm, or corroborate, the presence of the high-risk score related to blood pressure, which may represent blood pressure outside an acceptable range.

The method 360 may include updating or determining a patient score 370, for example, in response to corroborating the risk score 368 or in response to determining that further patient input is not needed 366. The patient score may be used to evaluate the overall condition of the patient.

In addition, the method 360 may include determining whether the patient is stable 372, for example, after administering treatment according to a current treatment regimen. Stability of the patient may be determined by comparing the overall patient score, one or more risk scores, one or more treatment scores, or one or more symptom scores, to a threshold value. In the illustrated embodiment, whether the patient is stable is related to whether one or more risk scores are within a stable region or within a non-stable region. In particular, the patient may be determined to be table or unstable based on whether one or more risk scores exceed a particular threshold value that defines the boundary between a stable region and a non-stable region.

The threshold value for stability may correspond to a threshold magnitude value of a score, a threshold rate of change of a score, or both. In one example, a patient who has a high but steady risk score may be considered unstable. In another example, a patient who has a low risk score that drastically increases but is still low, may also be considered unstable.

The method 360 may include determining a treatment regimen to stabilize risk scores 374, for example, in response to determining that the patient is not stable based on one or more risk scores. The treatment regimen to stabilize risk scores may prioritize the lowering or steadying (e.g., lowering the magnitude value or lowering the rate of change value) of risk scores over the lowering or steadying of other scores, such as treatment scores or symptom scores.

In some embodiments, the threshold value may be based on continuous functions used to generate the one or more scores and the function used to combine them into the patient score. Accordingly, the threshold value may be fixed or may be determined based on the relative value to other scores.

The method 360 may include determining a treatment regimen to optimize overall patient score 376, for example, in response to determining that the patient is stable based on one or more risk scores. The treatment regimen to optimize overall patient score may balance the risk scores, treatment scores, and symptom scores without necessarily prioritizing one type of score over the other. In some embodiments, when the patient is stable, the determined treatment regimen based on the treatment score may titrate of one or more treatments used in a previous treatment regimen, for example, toward a physician-based treatment regimen.

In addition, the method 360 may include providing the determined treatment regimen to administer treatment 378, for example, following determining the treatment regimen in 374 or 376. The treatment regimen may be provided to the treatment delivery system for administration.

Further, the method 360 may include confirming compliance with the administered treatment 380. For example, an image of the user's face may be recorded when medication is dispensed from a drug dispenser to confirm the identity of the patient. Such compliance data may be used by the method 360, for example, to update the patient score 370.

In some embodiments, the patient may not be stable 372 and the patient score may indicate that physician input is needed instead of management by the treatment management system. For example, an overall HF risk score may exceed a higher threshold level that indicates an HF event, which may suggest the patient should be seen in a hospital, emergency department, ambulance, observation unit, urgent care, or HF/cardiology clinic, by a nurse or physician. In such cases, the treatment management system may automatically notify the nurse or physician, as well as the patient, and may enter into an override mode to stop administration of treatment.

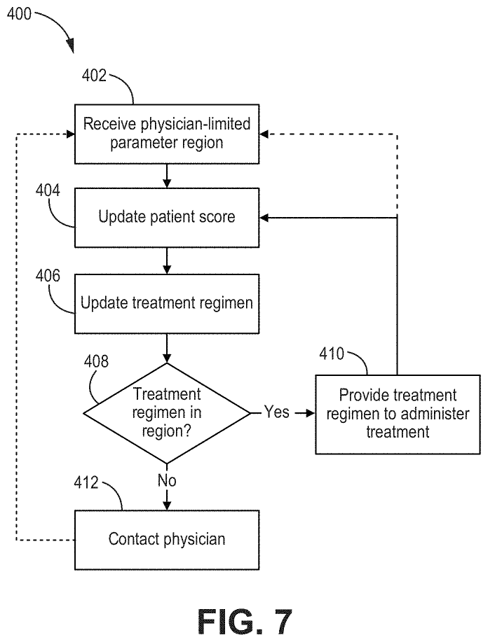

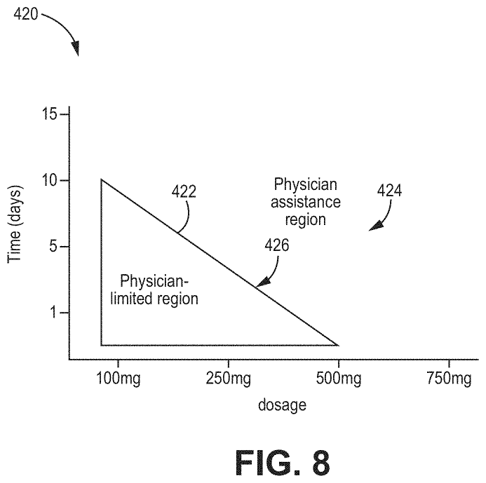

FIGS. 7-8 show the use of physician-limited treatment region to manage patient treatment. FIG. 7 is a flow diagram that illustrates one example of implementing the optimizing treatment method of FIG. 3 integrating physician input. FIG. 8 is a conceptual diagram that illustrates one example of a physician-limited parameter region usable in the method of FIG. 7.

As illustrated, a method 400 may include receiving a physician-limited parameter region 402 for a given patient. The physician-limited parameter region 402 may be different for different patients, different for different health statuses (e.g., HF class, NYHA II vs. NYHA III, etc.), and different for different treatments (e.g., specific medication or drug). The physician-limited parameter region may be based on physician input indicating an acceptable range of parameters that the treatment management system may use to administer treatment to the patient without further physician input, for example, between physician visits. The physician-limited parameter region may be stored in a memory of the treatment management system and may be described as a predetermined physician-limited, or physician-guided, parameter region.

The method 400 may include updating a patient score 404, for example, based on collected data. The patient score may be updated in the same or similar manner as updating the patient score 370 in FIG. 6.

The method 400 may also include updating a treatment regimen 406, for example, based on collected data. The treatment regimen may be updated in the same or similar manner as treatment regimen generation 326 in FIG. 4.

Once the treatment regimen is updated or determined, the method 400 may include determining whether the treatment regimen is within the physician-limited parameter region 402.

The determination may be based on a comparison of the treatment regimen with the physician-limited parameter region for one or more treatments. In some embodiments, a determination that the patient score, one or more risk scores, one or more treatment scores, or one or more symptoms scores may be made in addition to or as an alternative to directly comparing the treatment regimen with the physician-limited parameter region. For example, the physician-limited parameter region may include limitations on various scores or scores related to physician-limited treatment regimens may be calculated for comparison.

The method 400 may include providing the treatment regimen to the treatment delivery system to administer treatment 410, for example, in response to determining that the treatment regimen is within the physician-limited parameter region 408. The method 400 may return to updating the patient score 404 after administering treatment. The physician-limited parameter region may be again received, or updated, 402 each time the patient visits the physician.

Further, the method 400 may include initiating a process to contact the physician 412, for example, in response to determining that the treatment regimen is outside of the physician-limited parameter region 408 (e.g., in a physician assistance region). For example, a treatment regimen outside of the physician-limited parameter region may indicate that the treatment management system is unable to provide effective treatment of one or more risks or symptoms based on the available treatments in the physician-limited parameter region. The method 400 may return to receive an updated physician-limited parameter region 402.

One example of a physician-limited treatment region is shown in plot 420, which shows a physician-limited region 422 and a physician-assistance region 424. The physician-limited region 422 may also be described as a threshold region. The boundary 426 between the physician-limited region 422 and the physician assistance region 424 may also be described as a threshold.

The physician-limited region 422 may define one or more parameters for treatment that the treatment management system may operate within. Non-limiting examples of parameters that may be defined by the physician-limited region 422 include treatment dosage, dosage frequency, or cumulative treatment dosage over time.

In some embodiments, the physician-limited region 422 may take the shape of a triangle or other geometric shape, for example, when plotted for time versus dosage. As illustrated, a triangular shape may indicate that lower doses may be administered for a wider range of time compared to higher doses, which may only be allowed to be administered for a short period of time.

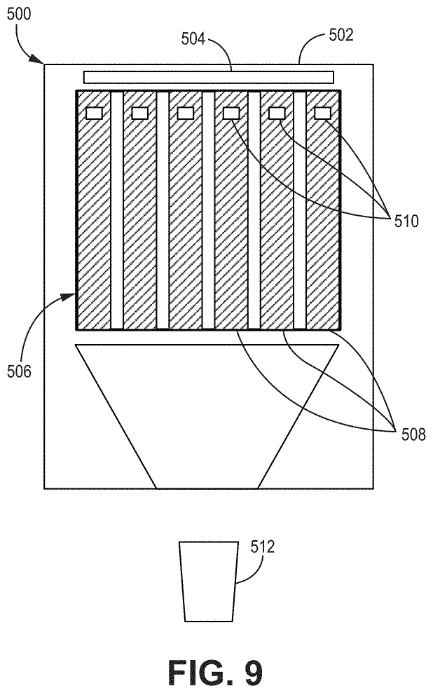

FIG. 9 is a conceptual diagram that illustrates one example of a treatment delivery system usable with the treatment management system of FIG. 1. As illustrated, FIG. 9 shows a treatment delivery system in the form of a drug dispenser 500. The drug dispenser 500 may include a housing 502 including one or more receptacles 506, or compartments. A communication interface 504 may be coupled to the housing 502. The one or more receptacles 506 are configured to receive or retain one or more cartridges 508. For example, one receptacle 506 may be configured to receive one cartridge 508 or multiple cartridges. In general, the drug dispenser 500 is configured to retain two or more cartridges 508.

Each cartridge 508 may include, or be coupled to, a unique drug identifier 510. The drug identifier 510 may be detectable by the communication interface 504 and may be used by the drug dispenser 500 to identify which drug is contained in each cartridge 508 and where the cartridge 508 is disposed in the one or more receptacles 506. For example, the drug identifier 510 may be an RFID tag that is detectable using an RFID transceiver of the communication interface 504. Other types of drug identifiers include optical encoders and electrical encoders.

In some embodiments, each cartridge 508 contains a different drug or a different dose. The drug dispenser 500 may be able to dispense a particular drug from a particular cartridge 508 by tracking the drug identifier 510 as indicated by the current treatment regimen. In other words, the cartridges 508 may be inserted into the drug dispenser 500 in any position or order, and the drug dispenser may still be able to distinguish the location of each cartridge. The dispensed drugs may be funneled into a single cup or other container 512 for the patient to use for taking the administered medication.

The communication interface 504 may also be configured to transmit a request to deliver one or more new cartridges 508. For example, the communication interface 504 may provide an order over the internet to initiate the delivery of particular cartridges 508 from a remote location to the home of the patient for local storage. The patient, or a delivery service, may insert the cartridges into the receptacle 506 of the drug dispenser 500. As each cartridge 508 is used, a new cartridge may be ordered, which may contain the same drug or dose as the empty cartridge or a different drug or dose depending on the requirements of the current treatment regimen.

In some embodiments, each cartridge 508 may be sealed and inaccessible to the patient or person loading the cartridge 508 into the receptacle 506. The cartridges 508 may only be accessible to the drug dispenser 500.

Each cartridge 508 may be configured to be inserted in a respective receptacle 506 to load the receptacle with the respective drug. In other embodiments, each cartridge 508 may be attached to the drug dispenser 500, and the drug dispenser may empty the drugs from the cartridge into the appropriate one or more receptacles 506 to load the receptacles. The drug dispenser 500 may track which receptacles are loaded with which drug based on the drug identifier 510 associated with the cartridge at the time of loading. In other words, the drug dispenser 500 may handle ensuring that each drug from each cartridge 508 reaches the appropriate receptacle 506. After being emptied, each cartridge 508 may be disposed.

The communication interface 504 may be configured to detect the drug identifier 510 before, at the same time, or after the respective cartridge 508 is loaded. For example, in some embodiments where the cartridge 508 is emptied to load a receptacle, the drug identifier 510 may be detected before or at the same time the cartridge 508 is attached to the drug dispenser 500.

FIG. 10 is a conceptual diagram that illustrates one example of managing treatment of a patient using the method of FIG. 3. As illustrated, a plot 600 shows how different treatment regimens are administered based on changes in a fluid level risk score (e.g., an impedance risk score). Time is plotted on the x-axis and dosage or fluid level is plotted on the y-axis in plot 600. In the illustrated example, the treatment management system adjusts treatment even when the fluid level risk score is in a stable region based on magnitude but not in a stable region based on rate of change (e.g., below the OptiVol™ high threshold such as 60 ohm-days but changing quickly) to maintain the patient in a steady and stable region. For example, when fluid levels increase at time 2, the treatment management system increases loop diuretics and electrolytes at time 2. When fluid levels decrease at time 3, the loop diuretics and electrolytes are decreased at time 4. When fluid levels increase again at time 5, the diuretics and electrolytes are also increased again at time 5. Other medications, such as ACE inhibitors and beta blockers may not change depending on the risk factors.

FIG. 11 is a conceptual diagram that illustrates another example of managing treatment of a patient using the method of FIG. 3. As illustrated, a plot 650 shows a diuretic treatment level, a renal failure risk score, a dietary sodium intake level, and an HF hospitalization risk score. Time is plotted on the x-axis and dosage or risk is plotted on the y-axis in plot 650. The treatment management system may administer treatment that balances various risks, such as fluid level and electrolyte level (e.g., sodium).

As shown, the heart failure hospitalization (AHF) risk score jumps at time 2, for example, due to increase in impedance from fluid. The treatment management system increases the amount of diuretic and electrolyte dosages at time 2 commensurate with the increased risk of AHF. In order to keep kidney failure risk score low, the treatment management system only increases the diuretic dosage for a short amount of time. As illustrated, when the AHF risk score drops at time 3, the diuretic dosage drops back down to low level at time 4 in order to maintain low kidney risk score. This balancing of risk scores and dosages may simultaneously keep the AHF risk score low and the kidney risk score low, as well as keep the patient on the lowest dosage as possible.

### Illustrative Embodiments

While the present disclosure is not so limited, an appreciation of various aspects of the disclosure will be gained through a discussion of the specific illustrative embodiments provided below. Various modifications of the illustrative embodiments, as well as additional embodiments of the disclosure, will become apparent herein.

In embodiment A1, a treatment management system comprises a sensor system to provide patient data about a patient or an environment related to the patient. The treatment management system also comprises a treatment delivery system to administer treatment based on a treatment regimen and to provide treatment compliance data. The treatment management system further comprises a treatment optimization system operably coupled to the sensor system and the treatment delivery system to update a treatment regimen based on the patient data from the sensor system and the treatment compliance data from the treatment delivery system and to provide the updated treatment regimen to the treatment delivery system.

In embodiment A2, a system comprises the system according to any A embodiment, wherein the treatment optimization system is configured to update the treatment regimen at least daily.

In embodiment A3, a system comprises the system according to any A embodiment, wherein the treatment optimization system is configured to update the treatment regimen automatically between physician inputs to maintain the patient in a stable region.

In embodiment A4, a system comprises the system according to any A embodiment, wherein the treatment optimization system is operably coupled to a remote system to receive related treatment data to train a risk score generator, a treatment regimen generator, or both, to update the treatment regimen.

In embodiment A5, a system comprises the system according to any A embodiment, wherein the treatment optimization system comprises an override mode to suspend the treatment regimen or the treatment delivery system comprises an override mode to suspend treatment administration.

In embodiment A6, a system comprises the system according to embodiment A5, wherein the treatment optimization system is configured to enter the override mode in response to physician input or environmental data.

In embodiment A7, a system comprises the system according to any A embodiment, wherein the treatment optimization system is operably coupled to the treatment delivery system over the internet or integrated into a single unit with the treatment delivery system.

In embodiment A8, a system comprises the system according to any A embodiment, wherein the treatment optimization system comprises a treatment regimen generator to provide the treatment regimen based on one or more risk scores.

In embodiment A9, a system comprises the system according to any A embodiment, wherein the treatment optimization system comprises a risk score generator to provide one or more risk scores based on patient data.

In embodiment A10, a system comprises the system according to any A embodiment, wherein at least one of the treatment delivery system or the treatment optimization system is configured to collect patient data to corroborate the one or more risk scores.

In embodiment A11, a system comprises the system according to any A embodiment, wherein the treatment optimization system comprises a treatment regimen generator to provide the treatment regimen based on a patient score determined based on at least one score selected from a risk score, a treatment score, and a symptom score.

In embodiment A12, a system comprises the system according to any A embodiment, wherein the sensor system comprises at least one of, a patient implantable sensor, a patient wearable sensor, an external sensor, a graphical user interface, or a memory to store historical patient data.

In embodiment A13, a system comprises the system according to embodiment A12, wherein the patient implantable sensor comprises at least one of an implantable electrical sensor comprising one or more electrical contacts, a biochemical sensor, a motion sensor, a piezoelectric sensor, an optical sensor, a temperature sensor, a geo-positioning sensor, or a microphone, operably coupled to circuitry of an implantable medical device.

In embodiment A14, a system comprises the system according to embodiment A12 or A13, wherein the patient wearable sensor comprises at least one of an external electrical sensor with one or more electrical contacts, a biochemical sensor, a motion sensor, a piezoelectric sensor, an optical sensor, a temperature sensor, a geo-positioning sensor, or a microphone.

In embodiment A15, a system comprises the system according to any embodiment A12-A14, wherein the external sensor comprises at least one of an imaging sensor, a weight scale, a pressure sensor, or microphone, operably coupled to circuitry of an external device.

In embodiment A16, a system comprises the system according to any A embodiment, wherein the treatment delivery system comprises at least one of a drug dispenser to contain one or more drugs, an automated treatment pump, or a graphical user interface to provide treatment information to the patient.

In embodiment A17, a system comprises the system according to any A embodiment, wherein the treatment delivery system comprises a processor to transmit a request over a communication interface to deliver one or more drugs from a remote location based on the treatment regimen to store locally in the treatment delivery system.

In embodiment B1, a treatment optimization system comprises a data communication interface operably couplable to a sensor system configured to provide patient data and a treatment delivery system configured to administer treatment based on a treatment regimen. The treatment optimization system also comprises a memory configured to store data representing a risk score generator and a treatment regimen generator and a processor operably coupled to the data communication interface and the memory. The processor is configured to: update a patient score in response to administering a previous treatment based on a previous treatment regimen, wherein the patient score is based on at least one score selected from the one or more risk scores, one or more treatment scores, and one or more symptom scores; determine a treatment regimen in response to the updated patient score and the previous treatment regimen; and provide the treatment regimen to the treatment delivery system.

In embodiment B2, a system comprises the system according to any B embodiment, wherein the treatment regimen is configured to minimize the patient score to minimize at least one of a risk score, a treatment deviation, or a patient symptom.

In embodiment B3, a system comprises the system according to any B embodiment, wherein the processor is further configured to update the patient score based on treatment compliance data.

In embodiment B4, a system comprises the system according to any B embodiment, wherein the processor is further configured to update the patient score based on patient corroboration of the one or more risk scores.

In embodiment B5, a system comprises the system according to any B embodiment, wherein the processor is further configured to update the patient score based on one or more monotonic functions, non-linear functions, or monotonic and non-linear functions, applied to the one or more risk scores.

In embodiment B6, a system comprises the system according to any B embodiment, wherein the treatment regimen comprises administration of a plurality of different treatments.

In embodiment B7, a system comprises the system according to any B embodiment, wherein the one or more symptom scores represent one or more patient symptoms determined based on sensor data or patient input.

In embodiment C1, a treatment optimization system comprises a data communication interface operably couplable to a sensor system configured to provide patient data and a treatment delivery system configured to administer treatment based on a treatment regimen. The communication interface is configured to receive data representing a predetermined physician-limited parameter region. The treatment optimization system also comprises a memory configured to store the data representing the predetermined physician-limited parameter region and a processor operably coupled to the data communication interface and the memory. The processor is configured to: determine a patient score based on at least one of a risk score, a treatment score, or a symptom score using the patient data; determine a treatment regimen based on the patient score; determine whether the treatment regimen is within the predetermined physician-limited parameter region; and provide the treatment regimen to the treatment delivery system in response to determining that the treatment regimen is within the predetermined physician-limited parameter region to administer treatment based on the treatment regimen.

In embodiment C2, a system comprises the system according to any C embodiment, wherein the processor is further configured to initiate a physician contact process in response to determining that the treatment regimen is outside of the predetermined physician-limited parameter region.

In embodiment C3, a system comprises the system according to any C embodiment, wherein the processor is configured to initiate a physician contact process in response to determining that one or more scores selected from a risk score, a treatment score, and a symptom score are outside of a threshold region.

In embodiment C4, a system comprises the system according to any C embodiment, wherein the predetermined physician-limited parameter region is based on at least one of treatment dosage, dosage frequency, or cumulative treatment dosage over time.

In embodiment D1, a treatment optimization system comprises a data communication interface operably couplable to a sensor system configured to provide patient data and a treatment delivery system configured to administer treatment based on a treatment regimen. The communication interface is configured to receive a physician-based treatment regimen. The treatment optimization system also comprises a memory configured to store data representing the physician-based treatment regimen and a processor operably coupled to the data communication interface and the memory. The processor is configured to: determine whether one or more risk scores are in a stable region representing a stable patient after administering treatment according to a current treatment regimen; determine a treatment score based on a difference between the current treatment regimen and the physician-based treatment regimen in response to the one or more risk scores being in the stable region; determine an updated treatment regimen based on the treatment score; and provide the updated treatment regimen to the treatment delivery system.

In embodiment D2, a system comprises the system according to any D embodiment, wherein the updated treatment regimen based on the treatment score is configured to minimize a patient score based on minimizing at least one of a risk score, a treatment score, or a symptom score.

In embodiment D3, a system comprises the system according to any D embodiment, wherein the updated treatment regimen based on the treatment score comprises titration of one or more treatments used in a previous treatment regimen.

In embodiment D4, a system comprises the system according to any D embodiment, wherein the processor is further configured to determine an updated treatment regimen based on the one or more risk scores in response to the one or more risk scores being in an unstable region representing an unstable patient.

In embodiment E1, the present disclosure comprises a treatment delivery system comprising a plurality of receptacles. Each receptacle is to retain a different drug or a different dose. The treatment delivery system also comprises one or more cartridges. Each cartridge is to contain a different drug. Each cartridge comprises a different drug identifier associated with a different drug or different dose to load into a respective receptacle. The treatment delivery system further comprises a controller configured to detect each drug identifier of the one or more cartridges and to transmit a request over a data communication interface to deliver a new cartridge associated with a specific drug identifier in response to an updated treatment regimen.

In embodiment E2, a system comprises the system according to any E embodiment, wherein the controller is further configured to deliver the request to a remote delivery system over the internet using the data communication interface.

In embodiment E3, a system comprises the system according to any E embodiment, further comprising the data communication interface further configured to detect the drug identifier associated with each cartridge and the specific receptacle where each cartridge is retained such that the new cartridge may be placed into any of the receptacles.

In embodiment E4, a system comprises the system according to any E embodiment, wherein the controller is further configured to load a drug in each of the one or more cartridges into a respective receptacle of the plurality of receptacles.

In embodiment E5, a system comprises the system according to any E embodiment, wherein each drug identifier comprises an RFID tag, optical encoder, or electrical encoder.

Thus, various embodiments of the ADAPTIVE TREATMENT MANAGEMENT SYSTEM are disclosed. Various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

All references and publications cited herein are each expressly incorporated herein by reference in their entirety for all purposes, except to the extent any aspect directly contradicts this disclosure.

All scientific and technical terms used herein have meanings commonly used in the art unless otherwise specified. The definitions provided herein are to facilitate understanding of certain terms used frequently herein and are not meant to limit the scope of the present disclosure.

The terms “coupled” or “connected” refer to elements being attached to each other either directly (in direct contact with each other) or indirectly (having one or more elements between and attaching the two elements). Either term may be modified by “operatively” and “operably,” which may be used interchangeably, to describe that the coupling or connection is configured to allow the components to interact to carry out functionality.

As used herein, the term “configured to” may be used interchangeably with the terms “adapted to” or “structured to” unless the content of this disclosure clearly dictates otherwise.

Th singular forms “a,” “an,” and “the” encompass embodiments having plural referents unless its context clearly dictates otherwise.

As used herein, “have,” “having,” “include,” “including,” “comprise,” “comprising” or the like are used in their open-ended sense, and generally mean “including, but not limited to.” It will be understood that “consisting essentially of” “consisting of,” and the like are subsumed in “comprising,” and the like.

Reference to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout are not necessarily referring to the same embodiment of the disclosure. Furthermore, the particular features, configurations, compositions, or characteristics may be combined in any suitable manner in one or more embodiments.

1. A heart failure treatment management system comprising:
a sensor system to provide patient data about a patient or an environment related to the patient using a non-implantable sensor and an implantable sensor;
a heart failure treatment delivery system to administer treatment based on a treatment regimen and to provide treatment compliance data; and
a heart failure treatment optimization system operably coupled to the sensor system and the heart failure treatment delivery system to update a heart failure treatment regimen based on the patient data from the sensor system and the treatment compliance data from the heart failure treatment delivery system and to provide the updated heart failure treatment regimen to the heart failure treatment delivery system.

2. The system according to claim 1, wherein the heart failure treatment optimization system is configured to update the treatment regimen at least daily.

3. The system according to claim 1, wherein the heart failure treatment optimization system is configured to update the treatment regimen automatically between physician inputs to maintain the patient in a stable region.

4. The system according to claim 1, wherein the heart failure treatment optimization system is operably coupled to a remote system to receive related treatment data to train a risk score generator, a treatment regimen generator, or both, to update the treatment regimen.

5. The system according to claim 1, wherein the heart failure treatment optimization system comprises an override mode to suspend the treatment regimen, the heart failure treatment delivery system comprises an override mode to suspend treatment administration, or both.

6. The system according to claim 1, wherein the heart failure treatment optimization system comprises a treatment regimen generator to provide the treatment regimen based on one or more risk scores.

7. The system according to claim 1, wherein the heart failure treatment optimization system comprises a risk score generator to provide one or more risk scores based on patient data.

8. The system according to claim 1, wherein at least one of the heart failure treatment delivery system or the heart failure treatment optimization system is configured to collect patient data to corroborate the one or more risk scores.

9. The system according to claim 1, wherein the heart failure treatment optimization system comprises a treatment regimen generator to provide the treatment regimen based on a patient score determined based on at least one score selected from:
a risk score indicating a decline in patient health;
a treatment score indicating a difference between a physician-based treatment regimen and a current treatment regimen being administered to the patient, and
a symptom score indicating patient symptoms not included in the patient data.

10. The system according to claim 1, wherein the non-implantable sensor of the sensor system comprises a patient wearable sensor.

11. The system according to claim 1, wherein the implantable sensor of the sensor system comprises one or more electrical contacts.

12. The system according to claim 11, wherein the external sensor comprises at least one of an imaging sensor, a perfusion sensor, a weight scale, a bed scale, a pressure sensor, or microphone, operably coupled to circuitry of an external device.

13. The system according to claim 1, wherein the treatment delivery system comprises at least one of a drug dispenser to contain one or more drugs, an automated treatment pump, a graphical user interface to provide treatment information to the patient, or an implantable electrical stimulator.

14. The system according to claim 1, wherein the treatment delivery system comprises a processor to transmit a request over a communication interface to deliver one or more drugs from a remote location based on the treatment regimen to store locally in the treatment delivery system.

15. A heart failure treatment optimization system comprising:
a data communication interface operably couplable to a sensor system configured to provide patient data and a treatment delivery system configured to administer treatment based on a treatment regimen;
a memory configured to store data representing a risk score generator and a treatment regimen generator; and
a processor operably coupled to the data communication interface and the memory, the processor configured to:
update a patient score in response to administering a previous treatment based on a previous treatment regimen, wherein the patient score is based on at least one score selected from the one or more risk scores, one or more treatment scores, and one or more symptom scores;
determine a treatment regimen in response to the updated patient score and the previous treatment regimen; and
provide the treatment regimen to the treatment delivery system.

16. The system according to claim 15, wherein the treatment regimen is configured to minimize the patient score to minimize at least one of a risk score, a treatment deviation, or a patient symptom.

17. The system according to claim 15, wherein the processor is further configured to update the patient score based on treatment compliance data.

18. The system according to claim 15, wherein the processor is further configured to update the patient score based on patient corroboration of the one or more risk scores.

19. The system according to claim 15, wherein the processor is further configured to determine the treatment regimen within the predetermined physician-limited parameter region, and initiate a physician contact process in response to determining that the treatment regimen is outside of the predetermined physician-limited parameter region.

20. A heart failure treatment delivery system comprising:
a plurality of receptacles, each receptacle to retain a different drug or a different dose;
one or more cartridges, each cartridge to contain a different drug, each cartridge comprising a different drug identifier associated with a different drug or different dose to load into a respective receptacle; and
a controller configured to detect each drug identifier of the one or more cartridges and to transmit a request over a data communication interface to deliver a new cartridge associated with a specific drug identifier in response to an updated treatment regimen.
